(19) United States
(12) Patent Application Publication
(10) Pub. No.: US 2018/0280696 A1
(43) Pub. Date: Oct. 4, 2018

(54) SYSTEMS, METHODS AND DEVICES FOR PREVENTING TISSUE DAMAGE

(71) Applicant: THE ARIZONA BOARD OF REGENTS on behalf of THE UNIVERSITY OF ARIZONA, Tucson, AZ (US)

(72) Inventors: Marvin J. Slepian, Tucson, AZ (US); David G. Armstrong, Tucson, AZ (US); Bijan Najafi, Tucson, AZ (US)

(21) Appl. No.: 15/762,626

(22) PCT Filed: Sep. 23, 2016
(86) PCT No.: PCT/US16/53359

§ 371 Date: Mar. 23, 2018

Related U.S. Application Data
(60) Provisional application No. 62/232,568, filed on Sep. 25, 2015.
(60) Provisional application No. 62/232,574, filed on Sep. 25, 2015.
(60) Provisional application No. 62/232,571, filed on Sep. 25, 2015.

(51) Int. Cl. A61N 1/36; A61B 5/103; A61B 5/0205; A61B 5/00; A61B 5/145; A61B 5/026; A61F 5/01; A61H 23/00; A61H 7/00; A43B 7/14

(52) U.S. Cl. CPC A61N 1/36031; A61H 2230/505; A61B 5/02055; A61B 5/443; A61B 5/14539; A61B 5/4875; A61B 5/442; A61B 5/4845; A61B 5/4266; A61B 5/026; A61F 5/0127; A61H 23/00; A61H 7/00; A61B 5/6807; A61B 5/447; A43B 7/141; A61B 2562/046; A61F 2005/0188; A61H 2201/02; A61H 2205/12; A61H 2230/255; A61B 5/1036

(57) ABSTRACT

A sensor interface device (e.g., orthotic device or immobilization device) is disclosed. More specifically, described is an individualized sensor device and system to prevent tissue damage resulting from, for example, pressure, torsion, temperature, shear, altered blood flow, reduced oxygen tension, pH levels, tissue conductivity, tissue viscoelasticity, infection and sweat chloride levels. In particular embodiments, the systems, devices and related methods are used for preventing ambulation and mobility related plantar tissue related damage and immobilizing tissues.

The present application claims priority to United States Provisional Patent Application Ser. Nos. 62/232,568, filed Sep. 25, 2015, 62/232,574, filed Sep. 25, 2015, and 62/232,571, filed Sep. 25, 2015, the entire disclosures of which are herein incorporated by reference in their entireties.

### FIELD OF THE INVENTION

A sensor interface device (e.g., orthotic device or immobilization device) is disclosed. More specifically, described is an individualized sensor device and system to prevent tissue damage resulting from, for example, pressure, torsion, temperature, shear, altered blood flow, reduced oxygen tension, pH levels, tissue conductivity, tissue viscoelasticity, infection and sweat chloride levels. In particular embodiments, the systems, devices and related methods are used for preventing ambulation and mobility related plantar tissue related damage and immobilizing tissues.

### BACKGROUND OF THE INVENTION

Contact between a subject and an exogenous surface may expose the tissue surface to physical, chemical, microbial or other agents, entities, processes or mechanisms that may be noxious. These noxious stimuli are stressors that may induce a reaction, damage, or other morbidities. Further, if an organism is ill the resilience to these interface exposures may be further compromised. For example in a patient with diabetic neuropathy and reduced sensation, prolonged stasis—e.g., laying in bed without movement, may induce pressure necrosis on the back, sacrum or over the iliac crest leading to ischemia, tissue breakdown, infection and decubitus formation. Another example—in the well individual with active ambulation e.g., walking, jogging and running, as well as with activities requiring continued or extended foot pressure, e.g., cycling, power rowing, extended periods (time) and force are exerted on the foot and more specifically the plantar surface. This continued pressure may result in tissue damage resulting from but not limited to pressure-mediated reduction in circulation with ischemia and vascular damage, tissue compaction and breakdown, shear-mediated tissue damage, altered temperature, reduced venous and lymphatic drainage and inoculation of the surface with contaminants, chemical mediators and microorganisms. Further in specific disease states this may be exacerbated.

With active ambulation e.g., walking, jogging and running, as well as with activities requiring continued or extended foot pressure, e.g., cycling, power rowing, extended periods (time) and force are exerted on the foot and more specifically the plantar surface. This continued pressure may result in tissue damage resulting from but not limited to pressure-mediated reduction in circulation with ischemia and vascular damage, tissue compaction and breakdown, shear-mediated tissue damage, altered temperature, reduced venous and lymphatic drainage and inoculation of the surface with contaminants, chemical mediators and microorganisms. Further in specific disease states this may be exacerbated.

In diabetes, for example, diabetic foot ulcer is a major complication of and specific form of tissue damage of the diabetic foot. It occurs in 15-25% of all patients with diabetes and precedes 84% of all diabetes-related lower-leg amputations (see, Singh, Armstrong, Lipsky, JAMA) e.g., Brem, et al., 2007 J. Clin. Inv. 117(5):1219-1222). A major increase in mortality among diabetic patients, observed over the past 20 years is considered to be due to the development of macro and micro vascular complications, including failure of the wound healing process. Wound healing is an innate mechanism of action that works reliably most of the time. A key feature of wound healing is stepwise repair of lost extracellular matrix (ECM) that forms the largest component of the dermal skin layer (see, e.g., Iakovos, et al., 2006 Wounds 18(7) 177-185). Controlled and accurate rebuilding is essential to avoid under- or over-healing that may lead to various abnormalities. But in some cases, certain disorders or physiological insult disturbs the wound healing process. Diabetes mellitus is one such metabolic disorder that impedes the normal steps of the wound healing process.

Should a diabetic patient develop a plantar ulcer, for whatever reason, treatment options are generally limited to a two-fold treatment plan. In the first instance, the prime objective is to obtain wound closure, which eliminates a portal of entry for bacterial invasion and development of limb-threatening infection. In the second instance, a further objective is to allow for a reduction in pressures on the foot or the “off-loading” of tissues (Armstrong, D. G., Lavery, L. A., Nixon, B. P. & Boulton, A. J. It's Not What You Put on, but What You Take Off: Techniques for Debriding and Off-Loading the Diabetic Foot Wound. Clin. Infect. Dis. 39 Suppl 2, S92-S99 (2004)). In this regard, protective orthopedic footwear has been shown to lower sited foot pressures and further has been shown to contribute to the healing and closing of wounds. Moreover, once a given plantar ulcer has been effectively closed, protective orthopedic footwear has been shown to prevent the recurrence of plantar ulcers (Ulbrecht, J. S., Hurley, T., Mauger, D. T. & Cavanagh, P. R. Prevention of Recurrent Foot Ulcers With Plantar Pressure-Based In-Shoe Orthoses: The CareFUL Prevention Multicenter Randomized Controlled Trial. Diabetes Care (2014)).

A number of factors guide the selection of the appropriate off-loading modality for a particular patient. A few of these factors are patient compliance, comfort, ease of application, and cost. Common methods of off-loading plantar ulcers are the use of total contact casts or lower leg walking boots.

Lower leg walking boots, also known as removable cast walkers, are often chosen in order to reduce application time and to allow the physician to have easy access to the wound site for wound care procedures. Exemplary walkers are disclosed in U.S. Pat. No. 5,078,128, granted January 1992, U.S. Pat. No. 5,329,705, granted July. 1994, and U.S. Pat. No. 5,378,223, granted Jan. 3, 1995, and in U.S. publication no. 2004/0019307, all assigned to Royce Medical Co. and all incorporated herein by reference. Walkers are usually quite easy to apply and remove, typically utilizing straps with VELCRO (hook and loop fasteners) or buckles.

However, the same ease with which a physician may remove the walker in order to inspect and treat the wound site also allows patients to remove the walker outside of the presence of the physician. Thus one concern with the use of walkers is that the healing of the ulcer will be severely compromised by patients removing the walker and ambulating without the product applied. A physician is thus left to wonder with each application of a walking boot whether the patient will follow the advice of the clinician or whether the healing will be compromised by the patient removing the walker. Studies done by members of this research team and others have suggested that patients with plantar wounds secondary to diabetes only wear their off-loading device for an average of 28 percent of their daily activity (Armstrong, D. G., Lavery, L. A., Kimbriel, H. R., Nixon, B. P. & Boulton, A. J. Activity Patterns of Patients With Diabetic Foot Ulceration: Patients with active ulceration may not adhere to a standard pressure off-loading regimen. Diabetes Care 26, 2595-2597 (2003)).

In comparison to the above, the total contact cast (TCC) has generally been considered the gold standard for off-loading plantar ulcers (Bus, S. A. et al. IWGDF Guidance on footwear and offloading interventions to prevent and heal foot ulcers in patients with diabetes. International Working Group on the Diabetic Foot (2015); Wu, S. C., Crews, R. T. & Armstrong, D. G. The pivotal role of offloading in the management of neuropathic foot ulceration. Curr. Diab. Rep. 5, 423-429 (2005)). The concept of utilizing the total contact cast to treat plantar ulcers was developed in the 1950's. The total contact cast must be applied and removed by a physician or a practitioner in a number of steps, as will be understood by the skilled artisan. The application is time consuming, since an inner shell of plaster must be applied and allowed to fully dry, and then an exterior shell of plaster must be applied. The exterior shell must typically be allowed to dry for a full 24 hours before a patient can put any weight on the TCC. Additionally, the TCC must be removed at least once every one to two weeks, if not more frequently, so that the physician can inspect and treat the plantar ulcerations. Of course, removal of the TCC requires another application of a TCC. Thus, it seems that the use of walkers is a more efficient and economic manner of treating plantar ulcerations.

While using removable cast walkers seems more feasible, there exist problems with employing both them and with TCCs.

Improved methods to detect tissue interface “stress” and “stressors” and to react to them to mitigate and/or elevate these stressors and risk exposure is sorely needed. In particular, improved methods for preventing ambulation, pressure and other force-mediated and related plantar tissue damage, including immobilization devices, for all individuals from well to disease afflicted are needed.

### SUMMARY OF THE INVENTION

The devices, systems, and methods described herein overcome problems with existing devices. In particular, the sensor interface and devices comprising the interface provide for both detection and reaction (e.g., alternations in local environment) that treat and prevent tissue injuries.

For example, in some embodiments, the present invention provides a sensor interface device comprising a skin-contact surface configured for engagement with a subject's skin, wherein the skin-contact surface is configured to a) measure skin-contact variables within a subject's skin or on a subject's skin engaged with the skin-contact surface, wherein the skin-contact variables are selected from pressure, torsion, shear, temperature, blood flow, oxygen tension, pH levels, tissue turgor, hydration, skin compliance, skin conductivity, tissue viscoelasticity, presence of offending microorganisms, contaminants, noxious materials, chemicals, and sweat constituents, and chloride levels; and b) identify regions within the subject's skin engaged with the skin-contact surface experiencing or at risk for experiencing altered levels of variables through a comparing of measured skin-contact variables with established norms for such measured skin-contact variables, wherein the established norms for such measured skin-contact variables are no risk for tissue damage, risk for experiencing tissue damage, and experiencing tissue damage; and c) implement a change within the structure of the skin-contact surface upon identification of a region within the subject's skin engaged with the skin-contact surface experiencing or at risk for experiencing altered levels of the variables, wherein such an implemented change within the structure of the skin-contact surface is configured to ameliorate the altered variable within the identified region.

In some embodiments, the sensor interface device further comprises one or more sensors configured to measure skin-contact variables within a subject's skin engaged with the skin-contact surface (e.g., sensors configured to measure pressure, temperature, blood flow, oxygen tension, pH levels, tissue fluid, tissue conductivity, tissue viscoelasticity or sweat chloride levels) within a subject's skin engaged with the skin-contact surface. In some embodiments, the sensor interface device further comprises a processor, wherein the processor is used for the comparing of measured skin-contact variables with established norms for such measured skin-contact variables occurs and the identification of regions within the subject's skin engaged with the skin-contact surface experiencing or at risk for experiencing tissue damage. In some embodiments, the processor utilizes one or more algorithms for such comparing of measured skin-contact variables with established norms for such measured skin-contact variables occurs and the identification of regions within the subject's skin engaged with the skin-contact surface experiencing or at risk for experiencing tissue damage. In some embodiments, the one or more algorithms are configured to a) receive the measured skin-contact variables obtained with the skin-contact surface, b) compare the received measured skin-contact variables with established norms for such measured skin-contact variables occurs, c) identify regions within the subject's skin engaged with the skin-contact surface experiencing or at risk for experiencing tissue damage based upon the comparison of the received measured skin-contact variables with established norms for such measured skin-contact variables occurs, and d) direct the skin-contact surface to implement a change within the structure of the skin-contact surface upon identification of a region within the subject's skin engaged with the skin-contact surface experiencing or at risk for experiencing tissue damage, wherein such an implemented change within the structure of the skin-contact surface is configured to ameliorate tissue damage within the identified region. In some embodiments, the established norm for no risk for tissue damage, the established norm for risk for experiencing tissue damage, and the established norm for experiencing tissue damage is specific for the subject. In some embodiments, the established norm for no risk for tissue damage, the established norm for risk for experiencing tissue damage, and the established norm for experiencing tissue damage is specific for a particular subject population. In some embodiments, the particular subject population is a subject population at risk for developing tissue damage resulting from one or more of pressure, temperature, blood flow, oxygen tension, pH levels, tissue fluid, tissue conductivity, tissue viscoelasticity and sweat chloride levels. In some embodiments, the particular subject population is a subject population having either type 1 or type 2 diabetes, a subject with compromised vascularity, a war fighter, or an athlete. In some embodiments, thee particular subject population is a subject population experiencing compromised vascularity, atherosclerosis, and/or sensation. In some embodiments, the comparing of measured skin-contact variables with established norms for such measured skin-contact variables indicates a region within the subject's skin engaged with the skin-contact surface as experiencing pressure within the established norm for risk for experiencing tissue damage or experiencing tissue damage, then the implemented change within the structure of the skin-contact surface is re-alignment of the skin-contact surface such that pressure on the skin of the subject is redistributed from the region within the subject's skin identified as experiencing pressure within the established norm for risk for experiencing tissue damage or experiencing tissue damage. In some embodiments, if the comparing of measured skin-contact variables with established norms for such measured skin-contact variables indicates a region within the subject's skin engaged with the skin-contact surface as experiencing a temperature within the established norm for risk for experiencing tissue damage or experiencing tissue damage, then the implemented change within the structure of the skin-contact surface is a change in temperature of the skin-contact surface such that the temperature on the skin of the subject is changed within the subject's skin identified as experiencing a temperature within the established norm for risk for experiencing tissue damage or experiencing tissue damage. In some embodiments, the change in temperature is an increase or reduction in temperature. In some embodiments, if the comparing of measured skin-contact variables with established norms for such measured skin-contact variables indicates a region within the subject's skin engaged with the skin-contact surface as experiencing blood flow within the established norm for risk for experiencing tissue damage or experiencing tissue damage, then the implemented change within the structure of the skin-contact surface is re-alignment of the skin-contact surface such that blood flow at the region within the subject's skin identified as experiencing blood flow within the established norm for risk for experiencing tissue damage or experiencing tissue damage is improved. In some embodiments, if the comparing of measured skin-contact variables with established norms for such measured skin-contact variables indicates a region within the subject's skin engaged with the skin-contact surface as experiencing oxygen tension at or above the established norm for risk for experiencing tissue damage or experiencing tissue damage, then the implemented change within the structure of the skin-contact surface is re-alignment of the skin-contact surface such that oxygen tension at the region within the subject's skin identified as experiencing oxygen tension, inflammation or change in temperature within the established norm for risk for experiencing tissue damage or experiencing tissue damage is improved. In some embodiments,

if the comparing of measured skin-contact variables with established norms for such measured skin-contact variables indicates a region within the subject's skin engaged with the skin-contact surface as experiencing pH at or above the established norm for risk for experiencing tissue damage or experiencing tissue damage, then the implemented change within the structure of the skin-contact surface is re-alignment of the skin-contact surface such that pH at the region within the subject's skin identified as experiencing pH within the established norm for risk for experiencing tissue damage or experiencing tissue damage is improved. In some embodiments, if
the comparing of measured skin-contact variables with established norms for such measured skin-contact variables indicates a region within the subject's skin engaged with the skin-contact surface as experiencing sweat chloride levels within the established norm for risk for experiencing tissue damage or experiencing tissue damage, then the implemented change within the structure of the skin-contact surface is re-alignment of the skin-contact surface such that sweat chloride levels at the region within the subject's skin identified as experiencing sweat chloride within the established norm for risk for experiencing tissue damage or experiencing tissue damage is improved. In some embodiments, the measuring, identifying and implementing occurs automatically or manually. In some embodiments, the device is configured for contact with a subject's skin or tissue or entire body. In some embodiments, the device is an article of clothing or a section thereof, a patch, bandage, wrap, shawl, stocking, sock, glove, hat, bed sheet, blanket, mattress, bed, seat, seat cushion, or pad. In some embodiments, the device is an orthotic device configured for contact with a subject's plantar region. In some embodiments, the
orthotic device is a part of a shoe, immobilization device or brace comprising a hollow shell, floor mat, bed, couch, carpet, car seat, and/or clothing. In some embodiments, the subject is a human subject. In some embodiments, the implemented change within the structure of the skin-contact surface is localized or systemic vibration or change in temperature, massage, pressure, or tension. In some embodiments, the implemented change within the structure of the skin-contact surface is localized or systemic electrical stimulation. In some embodiments, the implemented change within the structure of the skin-contact surface is localized or systemic re-alignment. In some embodiments, device comprises a plurality of detection zones (e.g., each comprising
a plurality of sensors). In some embodiments, the device comprises at least one (e.g., at least 2, at least 5, at least 10, at least 20, at least 50, or at least 100) sensors. In some embodiments, the device further comprises a plurality (e.g., at least 2, at least 5, at least 10, at least 20, at least 50, or at least 100) of reactive elements configured to implement the change. In some embodiments, the reactive means are, for example, selected from bladders configured to elevate and isolate one or more zones, polymeric, metallic or composite materials that change shape or configuration upon being activated and/or energized, electroactive polymers, thermoactive materials, nitinol, chemical means that perform reactions yielding physical material and compositional change, compressed gas, magnetic means, and rehydration of a desiccated material. In some embodiments, the device further comprises a component to send a signal from the detection component to the reactive means.

In some embodiments, the present invention provides a device comprising a hollow shell configured to envelop and immobilize a tissue region of a subject, wherein the hollow shell has an interior region and an exterior region, wherein at least a portion of the interior region comprises a skin-contact surface configured for engagement with a subject's skin, wherein the skin-contact surface is configured to a) measure skin-contact variables within a subject's skin engaged with the skin-contact surface, wherein the skin-contact variables are selected from, for example, pressure, temperature, blood flow, oxygen tension, pH levels, sweat chloride levels, inflammatory cytokines, antibodies, microbial content, tissue conductivity, tissue viscoelasticity, or biomarkers, b) identify a status of the subject's skin engaged with the skin-contact surface, wherein the status is selected from not experiencing tissue damage, at risk for experiencing tissue damage, and experiencing tissue damage, wherein the status is identified through a comparing of measured skin-contact variables with established norms for such measured skin-contact variables, wherein the established norms for such measured skin-contact variables are not experiencing tissue damage, at risk for experiencing tissue damage, and experiencing tissue damage; and c) communicate the identified status. In some embodiments, the hollow shell is configured to be secured onto the tissue region of the subject such that the skin-contact region is engaged with the tissue region of the subject requiring immobilization. In some embodiments, the skin-contact surface comprises one or more sensors configured to measure skin-contact variables within a subject's skin engaged with the skin-contact surface. In some embodiments, the one or more sensors are each configured to measure one or more of pressure, temperature, blood flow, oxygen tension, pH levels, sweat chloride levels, inflammatory cytokines, antibodies, tissue conductivity, tissue viscoelasticity, microbial content, or biomarkers within a subject's skin engaged with the skin-contact surface. In some embodiments, the one or more sensors are separately configured to measure one or more of pressure, temperature, blood flow, oxygen tension, pH levels, sweat chloride levels, inflammatory cytokines, antibodies, tissue conductivity, tissue viscoelasticity, microbial content, or biomarkers within a subject's skin engaged with the skin-contact surface. In some embodiments, the processor is used for the comparing of measured skin-contact variables with established norms for such measured skin-contact variables and the identification of regions within the subject's skin engaged with the skin-contact surface as not experiencing tissue damage, at risk for experiencing tissue damage or experiencing tissue damage. In some embodiments, the processor utilizes one or more algorithms for such comparing of measured skin-contact variables with established norms for such measured skin-contact variables and the identification of regions within the subject's skin engaged with the skin-contact surface as not experiencing tissue damage, at risk for experiencing tissue damage or experiencing tissue damage. In some embodiments, the one or more algorithms are configured to a) receive the measured skin-contact variables obtained with the skin-contact surface, b) compare the received measured skin-contact variables with established norms for such measured skin-contact variables, c) identify regions within the subject's skin engaged with the skin-contact surface as not experiencing tissue damage, at risk for experiencing tissue damage or experiencing tissue damage based upon the comparison of the received measured skin-contact variables with established norms for such measured skin-contact variables, and d) communicate the identified status. In some embodiments, the established norm for no risk for tissue damage, the established norm for risk for experiencing tissue damage, and the established norm for experiencing tissue damage is specific for the subject. In some embodiments, the established norm for no risk for tissue damage, the established norm for risk for experiencing tissue damage, and the established norm for experiencing tissue damage is specific for a particular subject population. In some embodiments, a particular subject population is a subject population at risk for developing tissue damage resulting from one or more of pressure, temperature, blood flow, oxygen tension, pH levels, sweat chloride levels, inflammatory cytokines, antibodies, tissue conductivity, tissue viscoelasticity, or biomarkers. In some embodiments, the particular subject population is a subject population having either type 1 or type 2 diabetes. In some embodiments, the particular subject population is a subject population experiencing compromised vascularity. In some embodiments, the subject is a human subject. In some embodiments, the skin-contact surface is configured to communicate the identified status via wireless communication, auditory communication, and/or visual communication. In some embodiments, the tissue region is an appendage of a subject or an extremity of a subject. In some embodiments, the tissue region is any tissue region of a subject experiencing or at risk for experiencing tissue damage (e.g., including or in the vicinity of a subject's foot, ankle, leg, knee, hip, back, rib, shoulder, neck, head, elbow, wrist, hand, or finger). In some embodiments, the tissue region comprises a tissue region including a broken bone. In some embodiments, the composition of the hollow shell is rubber, polyurethane, polyethylene, carbon, Kevlar, glass, or any mixture thereof In some embodiments, the hollow shell is pre-formed to generically fit a particular tissue region. In some embodiments, the hollow shell is custom designed to fit a particular tissue region specific for the subject. In some embodiments, the hollow shell is generated with a three-dimensional printer. In some embodiments, the hollow shell can be opened or closed. In some embodiments, the hollow shell is further configured that upon securing onto the tissue region of the subject, the hollow shell may be opened or closed. In some embodiments, when the hollow shell is opened, the tissue region requiring immobilization is exposed. In some embodiments, the hollow shell has thereon a removable portion. In some embodiments, the removable portion is positioned in the vicinity of the tissue region requiring immobilization. In some embodiments, the hollow shell is or has portions that are transparent. In some embodiments, the hollow shell has therein a drainage means. In some embodiments, the drainage means is one or more channels within the hollow shell having a closable opening to the exterior region of the hollow shell and a closable opening to the interior region of the hollow shell. In some embodiments, the hollow shell is configured such that application of a vacuum force to the one or more channels results in the drainage. In some embodiments, the hollow shell has therein a means for accessing the tissue region requiring immobilization while the hollow shell is secured. In some embodiments, the means is one or more channels within the hollow shell having an closable opening to the exterior region of the hollow shell and a closable opening to the interior region of the hollow shell. In some embodiments, a region of the device selected from, for example, the interior region, the plantar surface, the dorsal surface, the lateral surface, or the distal surface of the hollow shell is configured to pulsate. In some embodiments, the device is further configured to deliver negative pressure. In some embodiments, the pulsation is configured to remove tissue edema or infiltrate, stimulate vascular growth, remodeling angiogenesis, vasculogensis, and/or conduit genesis. In some embodiments, the device is further configured to deliver one or more agents selected from, for example, growth factors, angiogenic agents, antibiotics, orc anti-inflammatory agents. In some embodiments, the interior region of the hollow shell has thereon a desiccated hydrogel layer, wherein a hydrogel layer is generated upon rehydration of the desiccated hydrogel layer. In some embodiments, the device is further configured to deliver variable pressure (e.g., a range of variable pressure with a programmable sequence). In some embodiments, the device is further configured to deliver positive pressure (e.g., a range of positive pressure with a programmable sequence).

Further embodiments provide a system, comprising any of the aforementioned devices and a hydrogel or polymer, wherein the hydrogel or polymer is configured to be applied between a tissue region to be immobilized with the device and the interior region of the device. In some embodiments, the polymer is an elastomer or a foam. In some embodiments, the system further comprises an external processor configured to communicate with the device. In some embodiments, the external processor is configured to receive the identified status of the subject's skin engaged with the skin-contact surface.

Additional embodiments provide a system, comprising any of the aforementioned devices and an external processor configured to communicate with the device. In some embodiments, the external processor is configured to receive the identified status of the subject's skin engaged with the skin-contact surface.

Yet other embodiments provide a method of immobilizing a subject's tissue region, comprising a) immobilizing a subject's tissue region with any of the aforementioned devices; and b) applying a hydrogel between the interior region of the device and the subject's immobilized tissue region.

Still other embodiments provide a method of immobilizing a subject's tissue region, comprising a) immobilizing a subject's tissue region with any of the aforementioned devices; and b) generating a hydrogel layer between the interior region of the device and the subject's immobilized tissue region through rehydrating the desiccated hydrogel layer.

Additional embodiments are described herein.

### DESCRIPTION OF THE DRAWINGS

FIG. 1 shows an exemplary device of embodiments of the present invention. FIG. 1A shows a construct (sensor device) without a sensor. FIG. 1B shows a construct with a sensor. FIG. 1C shows a sensor device with a plurality of zones without sensors. FIG. 1D show a sensor device with a plurality of zones with sensors.

FIG. 2 exemplary methods steps of a sensor device of embodiments of the present invention. FIG. 2A shows a baseline before measurement of parameters. FIG. 2B shows identification of a region determined to be at risk. FIG. 2C shows identification of regions that react to modify or relieve risk. FIG. 2D shows a cross sectional view of an exemplary sensor device with identification of a region at risk and removal or pressure in regions at risk.

FIG. 3 shows an exemplary orthotic device of embodiments of the present invention. FIG. 3A shows a construct (orthotic device) without a sensor. FIG. 3B shows an orthotic device with a sensor. FIG. 3C shows an orthotic device with a plurality of zones without sensors. FIG. 3D show an orthotic device with a plurality of zones with sensors.

FIG. 4 exemplary methods steps of an orthotic device of embodiments of the present invention. FIG. 4A shows a baseline before measurement of parameters. FIG. 4B shows identification of a region determined to be at risk. FIG. 4C shows identification of regions that react to modify or relieve risk. FIG. 4D shows a cross sectional view of an exemplary orthotic device with identification of a region at risk and removal or pressure in regions at risk.

### DETAILED DESCRIPTION OF THE INVENTION

A sensor interface device (e.g., orthotic device or immobilization device) is disclosed. More specifically, described is an individualized sensor device and system to prevent tissue damage resulting from, for example, pressure, torsion, temperature, shear, altered blood flow, reduced oxygen tension, pH levels, tissue conductivity, tissue viscoelasticity, infection and sweat chloride levels. In particular embodiments, the systems, devices and related methods are used for preventing ambulation and mobility related plantar tissue related damage and immobilizing tissues.

The present invention addresses this need.

### Definitions

As used herein, the terms “sensor interface device” and “sensor devices” refer to devices that sense one or more variables (e.g., pressure, torsion, shear, temperature, blood flow, oxygen tension, pH levels, tissue turgor, hydration, material properties (e.g., compliance, conductivity, tissue viscoelasticity, presence of offending microorganisms, contaminants, noxious materials, chemicals, and sweat constituents, chloride levels) detected in skin or tissue (e.g., at the interface of the device and a surface of the skin or tissue) and identify regions that have levels of the variable outside of a normal or desired range (e.g., determined by an established norm). In some embodiments, sensor devices react to a level of a variable outside of the normal range and correct the level of the variable to a normal or desired range. In some embodiments, correcting the level of the variable treats or prevents a complication of a disease or condition.

As used herein, the term “orthotic device” refers to a sensor device designed to be used on a bottom surface of a foot (e.g., inserted in a shoe).

As used herein, the term “established norms for such measured skin-contact variables” refers to a normal or desired value of a variable measured using a device described herein. In some embodiments, the established norm are determined based on the average or typical value for a specific subject or a population average (e.g., for a general population or a specific population including the subject).

### I. Sensor Technology

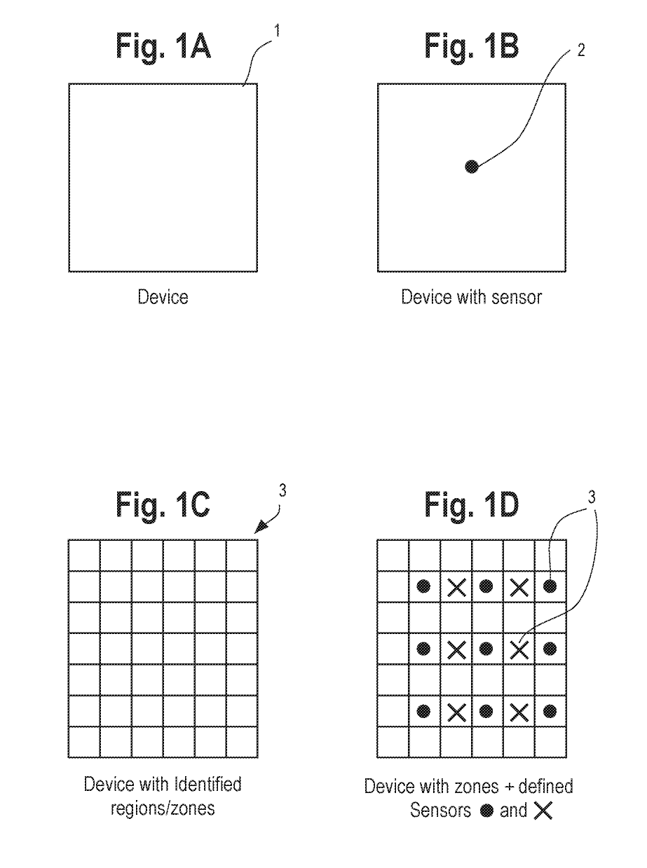

Provided herein are sensor devices and uses thereof. Exemplary devices are shown in FIGS. 1-4.

FIG. 1A shows construct (sensor device) 1 without sensor. FIG. 1B shows a sensor device with sensor 2. FIG. 1C-D shows an exemplary sensor device 1 comprising regions 3, without (1C) and with (1D) sensors 2.

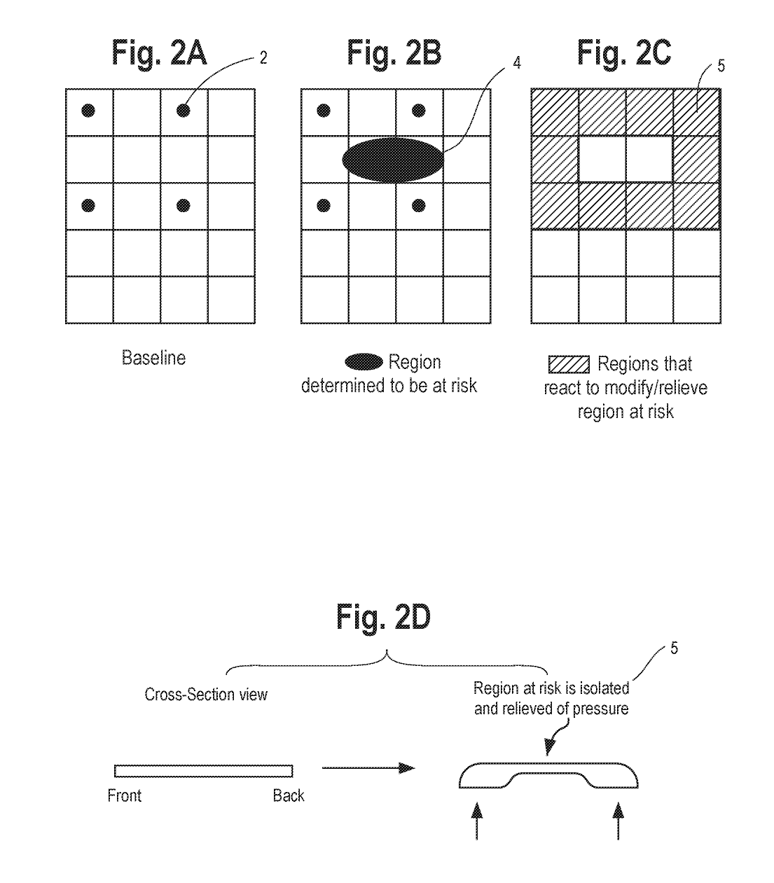

FIG. 2A-D shows an exemplary sensor device 1 in use. In FIG. 2A, sensor device 1 with regions 3 and sensors 2 is shown at baseline (e.g., prior to use). FIG. 2B shows detection of region 4 at risk of damage. FIG. 2C shows regions 5 that react to modify the region at risk and prevent damage. FIG. 2d shows a cross-section view of sensor device 1 showing region at risk 4.

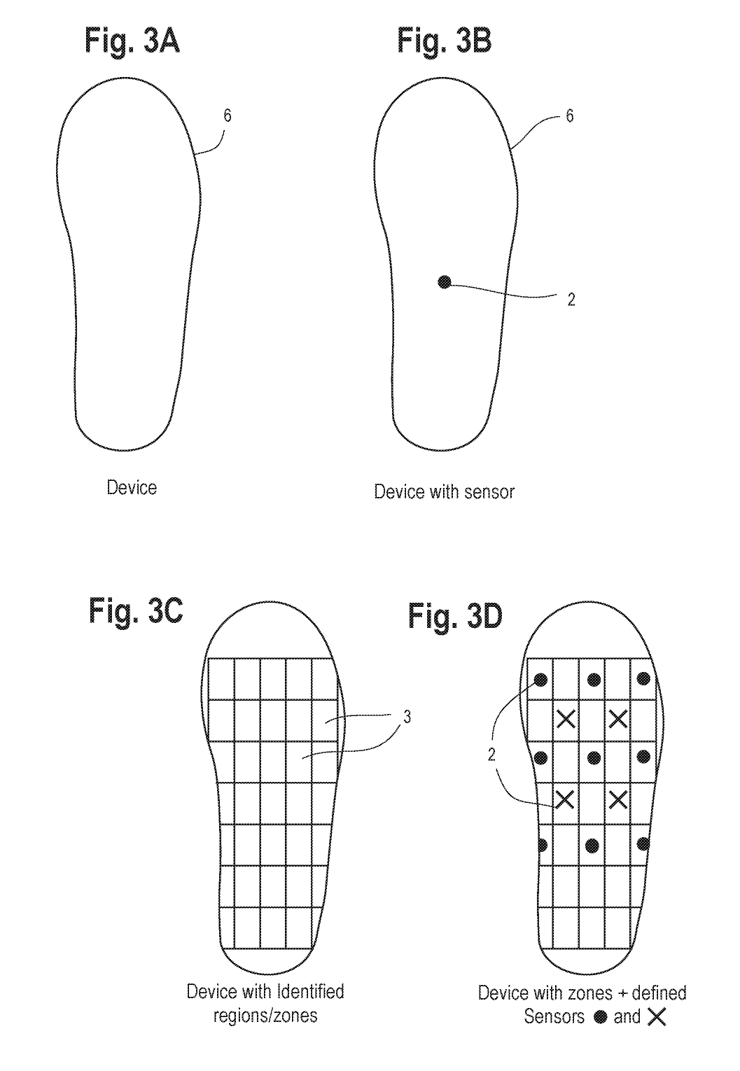

FIG. 3A shows construct (orthotic device) 6 without sensor. FIG. 6B shows an orthotic device 6 with sensor 2. FIG. 3C-D shows an exemplary orthotic device 6 comprising regions 3, without (3C) and with (3D) sensors 2.

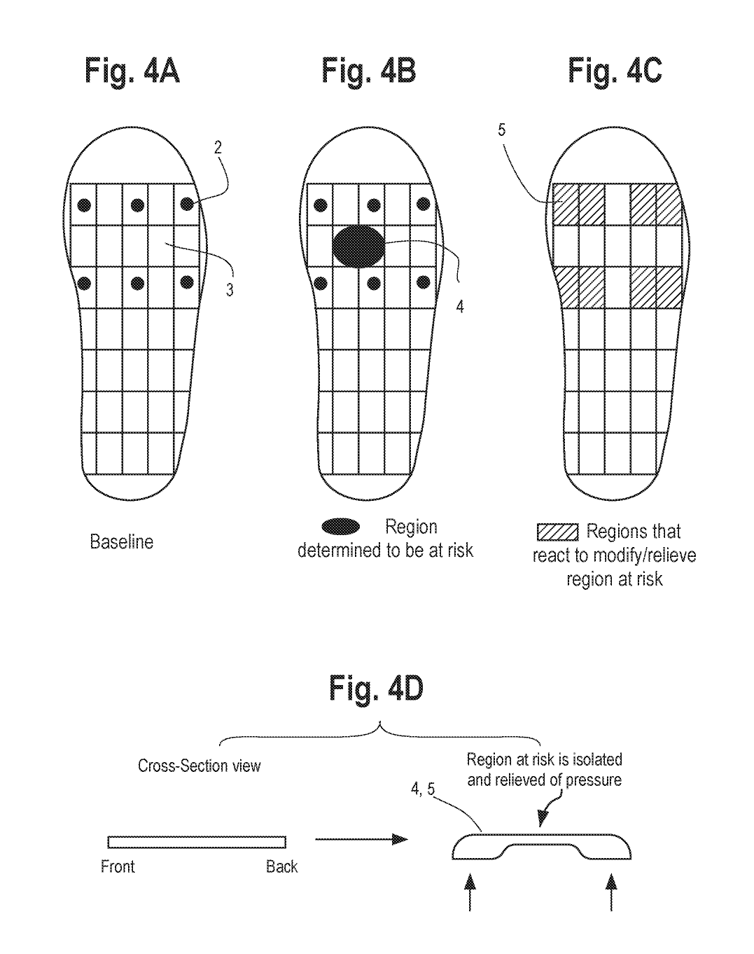

FIG. 4A-D shows an exemplary orthotic device 6 in use. In FIG. 4A, orthotic device 6 with regions 3 and sensors 2 is shown at baseline (e.g., prior to use). FIG. 4B shows detection of region 4 at risk of damage. FIG. 4C shows regions 5 that react to modify the region at risk and prevent damage. FIG. 4D shows a cross-section view of orthotic device 6 showing region at risk 4.

In particular, the devices of the present invention provides sensor (e.g., orthotic) devices comprising a skin-contact surface configured for engagement with a subject's skin or outer envelope tissue surface, wherein the skin-contact surface is configured to a) measure variables detectable at the device-skin/tissue interface, (both on the skin and within the skin) either singly or in combination, of a subject's skin engaged with the skin-contact surface, wherein the skin-contact variables are selected from pressure, torsion, shear, temperature, blood flow, oxygen tension, pH levels, tissue turgor, hydration, material properties (e.g., compliance, conductivity, tissue viscoelasticity, presence of offending microorganisms, contaminants, noxious materials, chemicals, and sweat constituents, chloride levels; b) identify regions on or within the subject's skin engaged with the skin-contact surface experiencing or at risk for experiencing tissue damage through a comparing of measured skin-contact variables with established norms for such measured skin-contact variables, wherein the established norms for such measured skin-contact variables are no risk for tissue damage, risk for experiencing tissue damage, and experiencing tissue damage; and c) implement a change within the structure of the skin-contact surface upon identification of a region within the subject's skin engaged with the skin-contact surface experiencing or at risk for experiencing tissue damage, wherein such an implemented change within the structure of the skin-contact surface is configured to ameliorate tissue damage within the identified region.

Such devices are not limited to a particular manner of measuring skin-contact variables. In some embodiments, the devices further comprise one or more sensors configured to measure extent of skin contact, degree of conformality, skin-contact variables on or within a subject's skin engaged with the skin-contact surface. The present invention is not limited to particular types or kinds of sensors. In some embodiments, the one or more (e.g., 2, 3, 4, 5, 10, 20, 50, 100, 1000, or more) sensors are each configured to measure pressure, temperature, shear, torsion, blood flow, oxygen tension, pH levels, microbial contamination, chemical contamination, tissue material properties, tissue conductivity, tissue viscoelasticity, sweat chloride and other sweat constituent levels on or within a subject's skin engaged with the skin-contact surface. In some embodiments, the one or more sensors are separately configured to measure pressure, temperature, shear, torsion, blood flow, oxygen tension, pH levels, microbial contamination, chemical contamination, tissue material properties, tissue conductivity, tissue viscoelasticity and sweat chloride and other sweat constituent levels within a subject's skin engaged with the skin-contact surface.

Other types of sensors may be used in addition to, or in place of, those described. For example, GPS sensors, microelectromechanical systems (MEMS) sensors, geomagnetic sensors, accelerometers, gyroscopes, or other types of sensors may be employed to provide movement or kinematic information that is unavailable from, or that is redundant to, other sensors.

In some embodiments, sensors are commercially available from any number of vendors.

In some embodiments, the device is divided into regions ranging from a single zone to a multiplicity of zones (e.g., 4, 8, 16, 32, 64, 128, or another number of zones). In some embodiments, each zone contains a single or multiple array or group of sensors. In some embodiments, the sensors function individually or in groups, or in various combinations. In some embodiments, the sensors are multiplexed. The zones serve to measure one or more of the parameters described above in discrete regions (e.g., singly or in a multiplex format).

In some embodiments, zones comprise a single or multiple types of reactive elements or means, to alter the progression, ameliorate or change the ongoing tissue response to detected tissue from the sensors (described further below and in FIGS. 2 and 4).

In some embodiments, reactive elements include, for example, contained bladders to elevate and isolate an afflicted zone (e.g., a zone detected or determined via the sensing elements or other means to be at risk for tissue damage or causing pain or discomfort). Other means of reactive change include, but are not limited to, reactive polymeric, metallic or composite materials that change shape or configuration upon being activated and/or energized, e.g. swelling with heating, bending with heating or chemically changing form, electroactive polymers, thermoactive materials, and nitinol. Other means of reactivity, actuation and “energizing” include chemical means (e.g., reactions yielding physical material and compositional change; compressed gas, magnetic means, rehydration of a desiccated material or any other means to physically alter the configuration of a regions or regions of the construct). The net dominant effect of all means of actuation is to physically deform and alter the extent/degree of conformity of the orthotic device surface with the apposed tissue (e.g., plantar foot surface).

In some embodiments, the devices further comprise a processor or are in a system comprising a process, computer (e.g., tablet, laptop, smart phone), and/or display screen. Such embodiments are not limited to a particular type or kind of processor. In some embodiments, the processor is used for the comparing of measured skin-contact variables with established norms for such measured skin-contact variables occurs and the identification of regions within the subject's skin engaged with the skin-contact surface experiencing or at risk for experiencing tissue damage. In some embodiments, the processor utilizes one or more algorithms for such comparing of measured skin-contact variables with established norms for such measured skin-contact variables occurs and the identification of regions within the subject's skin engaged with the skin-contact surface experiencing or at risk for experiencing tissue damage.

In some embodiments, the processor and system can function for full or partial on board data storage, data processing and telemetry. In some embodiments, data is directly downloaded or sent via telemetry (e.g., via near field, blue tooth, nested loops, etc.) to a user.

Such embodiments are not limited to particular algorithms. For example, in some embodiments, the one or more algorithms are configured to a) receive the measured skin-contact variables obtained with the skin-contact surface, b) compare the received measured skin-contact variables with established norms for such measured skin-contact variables occurs, c) identify regions within the subject's skin engaged with the skin-contact surface experiencing or at risk for experiencing tissue damage based upon the comparison of the received measured skin-contact variables with established norms for such measured skin-contact variables occurs, and d) direct the skin-contact surface to implement a change within the structure of the skin-contact surface upon identification of a region within the subject's skin engaged with the skin-contact surface experiencing or at risk for experiencing tissue damage, wherein such an implemented change within the structure of the skin-contact surface is configured to ameliorate tissue damage within the identified region, e.g., containing a single or range of reactive element or means.

Such embodiments are not limited to a particular established norm. For example, in some embodiments, the established norms are specific for the subject. In some embodiments, the established norms are specific for a patient population at risk for tissue related damage (e.g., for the cyclist—defined stress—e.g. time, pressure and temperature for a “century” ride or stage of a major race, for the diabetic population, individuals suffering from compromised vascularity, or the war fighter with extensive tissue stress (e.g., associated with a “long march.”). In some embodiments, the established norms are specific for athletes exerting high amounts of stress at a particular tissue region (e.g., bicyclists, runners, etc.). In some embodiments, the particular subject population is a subject population at risk for developing tissue damage resulting from one or more of pressure, temperature, shear, torsion, compaction, chemical or microbial contamination or exposure, altered blood flow, oxygen tension, pH levels, and sweat chloride and other constituents.

The devices of the present invention are not limited to particular implementation scenarios.

For example, in some embodiments, if the comparing of measured skin-contact variables with established norms for such measured skin-contact variables indicates a region on or within the subject's skin engaged with the skin-contact surface as experiencing pressure within the established norm for risk for experiencing tissue damage or experiencing tissue damage, then the implemented change within the structure of the skin-contact surface is re-alignment of the skin-contact surface such that pressure on the skin of the subject is redistributed from the region within the subject's skin identified as experiencing pressure within the established norm for risk for experiencing tissue damage or experiencing tissue damage.

In some embodiments, if the comparing of measured skin-contact variables with established norms for such measured skin-contact variables indicates a region on or within the subject's skin engaged with the skin-contact surface as experiencing a temperature within the established norm for risk for experiencing tissue damage or experiencing tissue damage, then the implemented change within the structure of the skin-contact surface is a change in temperature of the skin-contact surface such that the temperature on the skin of the subject is changed (e.g., increased or decreased) within the subject's skin identified as experiencing a temperature within the established norm for risk for experiencing tissue damage or experiencing tissue damage.

In some embodiments, if the comparing of measured skin-contact variables with established norms for such measured skin-contact variables indicates a region on or within the subject's skin engaged with the skin-contact surface as experiencing blood flow within the established norm for risk for experiencing tissue damage or experiencing tissue damage, then the implemented change within the structure of the skin-contact surface is re-alignment of the skin-contact surface such that blood flow at the region within the subject's skin identified as experiencing blood flow within the established norm for risk for experiencing tissue damage or experiencing tissue damage is improved.

In some embodiments, if the comparing of measured skin-contact variables with established norms for such measured skin-contact variables indicates a region on or within the subject's skin engaged with the skin-contact surface as experiencing oxygen tension at or above the established norm for risk for experiencing tissue damage or experiencing tissue damage, then the implemented change within the structure of the skin-contact surface is re-alignment of the skin-contact surface such that oxygen tension at the region within the subject's skin identified as experiencing oxygen tension within the established norm for risk for experiencing tissue damage or experiencing tissue damage is improved.

In some embodiments, if the comparing of measured skin-contact variables with established norms for such measured skin-contact variables indicates a region on or within the subject's skin engaged with the skin-contact surface as experiencing pH at or above the established norm for risk for experiencing tissue damage or experiencing tissue damage, then the implemented change within the structure of the skin-contact surface is re-alignment of the skin-contact surface such that pH at the region within the subject's skin identified as experiencing pH within the established norm for risk for experiencing tissue damage or experiencing tissue damage is improved.

In some embodiments, if the comparing of measured skin-contact variables with established norms for such measured skin-contact variables indicates a region on or within the subject's skin engaged with the skin-contact surface as experiencing sweat chloride levels within the established norm for risk for experiencing tissue damage or experiencing tissue damage, then the implemented change within the structure of the skin-contact surface is re-alignment of the skin-contact surface such that sweat chloride levels at the region within the subject's skin identified as experiencing sweat chloride within the established norm for risk for experiencing tissue damage or experiencing tissue damage is improved.

Similarly, the devices are not limited to a particular implemented change. In some embodiments, the implemented change within the structure of the skin-contact surface is localized or systemic vibration. In some embodiments, the implemented change within the structure of the skin-contact surface is localized or systemic undulation. In some embodiments, the implemented change within the structure of the skin-contact surface is localized or systemic electrical stimulation. In some embodiments, the implemented change within the structure of the skin-contact surface is localized or systemic re-alignment.

In some embodiments, the measuring, identifying and implementing occurs automatically. In some embodiments, the implementing occurs either automatically or manually.

In certain embodiments, the present invention provides systems comprising such an device and a processor. In some embodiments, the systems further comprise a visual display (e.g., a computer display, a smart phone, a smart tablet, etc.).

In certain embodiments, the devices are configured to store information obtained with the device.

In certain embodiments, the system has a display means to identify regions of the detected variable. In some embodiments, the display is physical (e.g., a small screen or hand held or wearable device or watch or virtual) telemetered or otherwise sent to a display device (e.g., computer, phone, watch or the like. In some embodiments, the display has the outline of the contacting surface, e.g. the sole of the foot. In some embodiments, regional information is displayed of single or multiple variables. In some embodiments, the ability to send back a signal to create change in surrounding regions, to an identified hot spot is described and is contained within the system. The system, as outlined above, is able to react in one or a multiplicity of zones, either automatically or manually.

### II. Support and Orthotic Devices

In some embodiments, the sensor interface devices and systems described herein are utilized in a support or orthotic device. In some exemplary embodiments, the devices and systems described herein are used in the treatment and prevention of wounds.

As described above, the TCC is the gold standard for treating ulcers, however, very few people actually use the gold-standard TCC (fewer than 2% of patients receive the TCC as their primary means of offloading) (Wu, S. C., Jensen, J. L., Weber, A. K., Robinson, D. E. & Armstrong, D. G. Use of pressure offloading devices in diabetic foot ulcers: do we practice what we preach. Diabetes Care 31, 2118-2119 (2008)). In addition, The RCW offloads as well as the TCC in the lab (Lavery, L. A., Vela, S. A., Lavery, D. C. & Quebedeaux, T. L. Reducing dynamic foot pressures in high-risk diabetic subjects with foot ulcerations. A comparison of treatments. Diabetes Care 19, 818-821 (1996)) but the RCW does not heal people as well as the TCC in randomized studies (Armstrong, D. G. et al. Offloading the diabetic foot wound: a randomized clinical trial (Abstract). Diabetes 50, A76 (2001)). The RCW fails largely because people are not wearing the device (Armstrong, D. G., Lavery, L. A., Kimbriel, H. R., Nixon, B. P. & Boulton, A. J. Activity Patterns of Patients With Diabetic Foot Ulceration: Patients with active ulceration may not adhere to a standard pressure off-loading regimen. Diabetes Care 26, 2595-2597 (2003)). For this reason, embodiments of the present disclosure provide a technique to render the removable (Armstrong, D. G. et al. Technique for Fabrication of an “Instant Total-Contact Cast” for Treatment of Neuropathic Diabetic Foot Ulcers. J. Am. Podiatr. Med. Assoc. 92, 405-408 (2002)) RCW irremovable. These devices behave better than its removable counterpart and about as well as the gold standard (Katz, I. A. et al. A randomized trial of two irremovable off-loading devices in the management of plantar neuropathic diabetic foot ulcers. Diabetes Care 28, 555-559 (2005); Armstrong, D. G., Lavery, L. A., Wu, S. & Boulton, A. J. M. Evaluation of Removable and Irremovable Cast Walkers in the Healing of Diabetic Foot Wounds: a Randomized Controlled Trial. Diabetes Care 28, 551-554 (2005)).

While removable walkers rendered irremovable and TCCs appear to be useful techniques for managing this high-risk patient population, covering a wound in a neuropathic extremity (at high risk for amputation) for days or even weeks inspires neither confidence nor information to the clinician. There exist a body of evidence that indicates that tracking wound analytes such as inflammatory cytokines, temperature, pH and other materials are an effective means of identifying potential risk for wound deterioration and infection (Armstrong, D. G. & Giovinco, N. A. Diagnostics, theragnostics, and the personal health server: fundamental milestones in technology with revolutionary changes in diabetic foot and wound care to come. Foot Ankle Spec. 4, 54-60 (2011); Armstrong, D. G., Lew, E. J., Hurwitz, B. & Wild, T. The Quest for Tissue Repair's Holy Grail: The Promise of Wound Diagnostics or Just another Fishing Expedition. Wound Medicine).

Accordingly, in some embodiments, provided herein are improved devices and system that meet the unmet need for treating and preventing wounds. In some embodiments, the devices described herein allow easy access to wounds on the plantar surface of the foot without removal and reapplication of the entire device. The devices have added comfort, providing the immobilization of a TCC with a more universal outside shell or boot configuration that may be filled on an individual basis, like a mold-cast, to provide the TCC effect.

The wound may be any type of wound, or damaged area of tissue, and may include wounds from trauma, surgery, or other causes, such as a diabetic ulcer. The tissue site, which includes the wound, may be the bodily tissue of any human, animal, or other organism, including bone tissue, adipose tissue, muscle tissue, dermal tissue, vascular tissue, connective tissue, cartilage, tendons, ligaments, or any other tissue. Treatment of the tissue site may include removal of fluids, e.g., exudate, or delivery of reduced pressure.

In some embodiments, devices immobilize the plantar surface for force (stress, i.e. force/area) distribution, while at the same time allowing for comfort of the distal leg and upper foot.

In some embodiments, devices (e.g., boots) comprise portals, windows or means for opening to allow access to the plantar surface of the foot, to the effected ulcer area, the adjacent area or any area of the foot/leg involved. The means for opening include, but are not limited to, multiple components of boot that attach together, e.g., a hinged system, screws, fasteners, a polymer window that is removed.

In some embodiments, devices comprise a drainage component to remove fluid, blood, exudate, sweat, other effluent, pus, etc. In some embodiments, devices incorporate a vacuum (e.g., negative or subatmospheric pressure means) to act as a wound vacuum.

In some embodiments, devices incorporate a pulsation component to mobilize edema, exudate and infiltrates. In some embodiments, pulsation is used independently or in a set synchromy with negative pressure. In some embodiments, pulsation adds the advantage of therapeutic vessel recruitment, remodeling and growth. The majority of wounds suffer from inadequate vascularization as a result of atherosclerosis or microvascular disease. Pulsation has been demonstrated to induce recruitment and growth of blood vessels, reducing ischemia, enhancing oxygenation and improving healing (18-24). In some embodiments, pulsation is global in the device (e.g., utilized with a defined or variable frequency or compartmentalized to regional domains in the device). This is termed local conditioning or precondition or reconditioning.

The devices described herein find use in feet and any other appendage (e.g., arm, etc.) or body part.

In some embodiments, the devices are orthotic devices. In some embodiments, orthotic devices are insoles, arch supports, etc. In some embodiments, the orthotic device is inserted into footwear. In some embodiments, an orthotic device is configured for contact with a subject's plantar region.

In some embodiments, the device is constructed of any suitable material (e.g., high density or ultra-high density memory foam, silicone, latex, neoprene, plastizote, Poron, ethylene vinyl acetate (EVA), polyethylene (PE) foam, polyurethane (PU) foam, polycarbonate, metal, etc.). In some embodiments, the device comprises structural and cushioning layers. In some embodiments, an anti-fungal, anti-microbial and anti-sweat top cloth may be laminated to the top layer of the device.

In some embodiments, the orthotic device is a part of a shoe, floor mat, carpet, and/or car seat. In some embodiments, the orthotic device is configured to be received within a shoe.

In certain embodiments, the present invention provides methods for preventing ambulation related tissue damage through use of an device (e.g., orthotic device) of the present invention through incorporation of such a device within the subject's shoes.

The devices described herein allow for protection of a tissue or body part while providing real-time feedback as to the status of a wound or other parameter. The present invention is not limited to the devices, systems, and methods described herein. Additional embodiments and uses are specifically contemplated.

All publications and patents mentioned in the above specification are herein incorporated by reference as if expressly set forth herein. Various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the invention.

What is claimed:

1. A sensor interface device comprising a skin-contact surface configured for engagement with a subject's skin, wherein the skin-contact surface is configured to
a) measure skin-contact variables within a subject's skin or on a subject's skin engaged with the skin-contact surface, wherein the skin-contact variables are selected from pressure, torsion, shear, temperature, blood flow, oxygen tension, pH levels, tissue turgor, hydration, skin compliance, skin conductivity, tissue viscoelasticity, presence of offending microorganisms, contaminants, noxious materials, chemicals, and sweat constituents, and chloride levels; and
b) identify regions within the subject's skin engaged with the skin-contact surface experiencing or at risk for experiencing altered levels of said variables through a comparing of measured skin-contact variables with established norms for such measured skin-contact variables, wherein the established norms for such measured skin-contact variables are no risk for tissue damage, risk for experiencing tissue damage, and experiencing tissue damage; and
c) implement a change within the structure of the skin-contact surface upon identification of a region within the subject's skin engaged with the skin-contact surface experiencing or at risk for experiencing altered levels of said variables, wherein such an implemented change within the structure of the skin-contact surface is configured to ameliorate said altered variable within the identified region.

2. The sensor interface device of claim 1, further comprising one or more sensors configured to measure skin-contact variables within a subject's skin engaged with the skin-contact surface.

3. The sensor interface device of claim 2, wherein the one or more sensors are each configured to measure pressure, temperature, blood flow, oxygen tension, pH levels, tissue fluid, tissue conductivity, tissue viscoelasticity and sweat chloride levels within a subject's skin engaged with the skin-contact surface.

4. (canceled)

5. The sensor interface device of claim 1, further comprising a processor, wherein the processor is used for the comparing of measured skin-contact variables with established norms for such measured skin-contact variables occurs and the identification of regions within the subject's skin engaged with the skin-contact surface experiencing or at risk for experiencing tissue damage.

6. The sensor interface device of claim 5, wherein the processor utilizes one or more algorithms for such comparing of measured skin-contact variables with established norms for such measured skin-contact variables occurs and the identification of regions within the subject's skin engaged with the skin-contact surface experiencing or at risk for experiencing tissue damage.

7. The sensor interface device of claim 6, wherein the one or more algorithms are configured to
a) receive the measured skin-contact variables obtained with the skin-contact surface,
b) compare the received measured skin-contact variables with established norms for such measured skin-contact variables occurs,
c) identify regions within the subject's skin engaged with the skin-contact surface experiencing or at risk for experiencing tissue damage based upon the comparison of the received measured skin-contact variables with established norms for such measured skin-contact variables occurs, and
d) direct the skin-contact surface to implement a change within the structure of the skin-contact surface upon identification of a region within the subject's skin engaged with the skin-contact surface experiencing or at risk for experiencing tissue damage, wherein such an implemented change within the structure of the skin-contact surface is configured to ameliorate tissue damage within the identified region.

8-10. (canceled)

11. The sensor interface device of claim 10, wherein the particular subject population is a subject population having either type 1 or type 2 diabetes, a subject with compromised vascularity, a war fighter, or an athlete.

12. The sensor interface device of claim 10, wherein the particular subject population is a subject population experiencing compromised vascularity, atherosclerosis, and/or sensation.

13. The sensor interface device of claim 1, wherein if the comparing of measured skin-contact variables with established norms for such measured skin-contact variables indicates a region within the subject's skin engaged with the skin-contact surface as experiencing pressure within the established norm for risk for experiencing tissue damage or experiencing tissue damage, then the implemented change within the structure of the skin-contact surface is re-alignment of the skin-contact surface such that pressure on the skin of the subject is redistributed from the region within the subject's skin identified as experiencing pressure within the established norm for risk for experiencing tissue damage or experiencing tissue damage.

14. The sensor interface device of claim 1, wherein if the comparing of measured skin-contact variables with established norms for such measured skin-contact variables indicates a region within the subject's skin engaged with the skin-contact surface as experiencing a temperature within the established norm for risk for experiencing tissue damage or experiencing tissue damage, then the implemented change within the structure of the skin-contact surface is a change in temperature of the skin-contact surface such that the temperature on the skin of the subject is changed within the subject's skin identified as experiencing a temperature within the established norm for risk for experiencing tissue damage or experiencing tissue damage.

15-16. (canceled)

17. The sensor interface device of claim 1, wherein if the comparing of measured skin-contact variables with established norms for such measured skin-contact variables indicates a region within the subject's skin engaged with the skin-contact surface as experiencing blood flow within the established norm for risk for experiencing tissue damage or experiencing tissue damage, then the implemented change within the structure of the skin-contact surface is re-alignment of the skin-contact surface such that blood flow at the region within the subject's skin identified as experiencing blood flow within the established norm for risk for experiencing tissue damage or experiencing tissue damage is improved.

18. The sensor interface device of claim 1, wherein if the comparing of measured skin-contact variables with established norms for such measured skin-contact variables indicates a region within the subject's skin engaged with the skin-contact surface as experiencing oxygen tension at or above the established norm for risk for experiencing tissue damage or experiencing tissue damage, then the implemented change within the structure of the skin-contact surface is re-alignment of the skin-contact surface such that oxygen tension at the region within the subject's skin identified as experiencing oxygen tension, inflammation or change in temperature within the established norm for risk for experiencing tissue damage or experiencing tissue damage is improved.

19. The sensor interface device of claim 1, wherein if the comparing of measured skin-contact variables with established norms for such measured skin-contact variables indicates a region within the subject's skin engaged with the skin-contact surface as experiencing pH at or above the established norm for risk for experiencing tissue damage or experiencing tissue damage, then the implemented change within the structure of the skin-contact surface is re-alignment of the skin-contact surface such that pH at the region within the subject's skin identified as experiencing pH within the established norm for risk for experiencing tissue damage or experiencing tissue damage is improved.

20. The sensor interface device of claim 1, wherein if the comparing of measured skin-contact variables with established norms for such measured skin-contact variables indicates a region within the subject's skin engaged with the skin-contact surface as experiencing sweat chloride levels within the established norm for risk for experiencing tissue damage or experiencing tissue damage, then the implemented change within the structure of the skin-contact surface is re-alignment of the skin-contact surface such that sweat chloride levels at the region within the subject's skin identified as experiencing sweat chloride within the established norm for risk for experiencing tissue damage or experiencing tissue damage is improved.

21-23. (canceled)

24. The sensor interface device of claim 1, wherein the device is an article of clothing or a section thereof, a patch, bandage, wrap, shawl, stocking, sock, glove, hat, bed sheet, blanket, mattress, bed, seat, seat cushion, or pad.

25. (canceled)

26-28. (canceled)

29. The sensor interface device of claim 1, wherein the implemented change within the structure of the skin-contact surface is localized or systemic vibration, change in temperature, massage, pressure, or tension, localized or systemic electrical stimulation, or localized or systemic re-alignment.

30-32. (canceled)

33. The sensor interface device of claim 1, wherein said device comprises a plurality of detection zones.

34-68. (canceled)

69. A system, comprising a sensor interface device of claim 1 and a processor.

70-75. (canceled)

76. An orthotic device comprising a skin-contact surface configured for engagement with a subject's skin, wherein the skin-contact surface is configured to
a) measure skin-contact variables within a subject's skin or on a subject's skin engaged with the skin-contact surface, wherein the skin-contact variables are selected from pressure, torsion, shear, temperature, blood flow, oxygen tension, pH levels, tissue turgor, hydration, skin compliance, skin conductivity, tissue viscoelasticity, presence of offending microorganisms, contaminants, noxious materials, chemicals, and sweat constituents, and chloride levels, wherein said skin is on at least a portion of the subject's foot; and
b) identify regions within the subject's skin engaged with the skin-contact surface experiencing or at risk for experiencing tissue damage through a comparing of measured skin-contact variables with established norms for such measured skin-contact variables, wherein the established norms for such measured skin-contact variables are no risk for tissue damage, risk for experiencing tissue damage, and experiencing tissue damage; and
c) implement a change within the structure of the skin-contact surface upon identification of a region within the subject's skin engaged with the skin-contact surface experiencing or at risk for experiencing tissue damage, wherein such an implemented change within the structure of the skin-contact surface is configured to ameliorate tissue damage within the identified region.

77-81. (canceled)
